(19) United States
(12) Patent Application Publication
(10) Pub. No.: US 2018/0313855 A1
(43) Pub. Date: Nov. 1, 2018

(54) METHODS FOR QUANTIFYING TOTAL VITAMIN D

(71) Applicant: Waters Technologies Corporation, Milford, MA (US)

(72) Inventors: Jinchuan Yang, Hopkinton, MA (US); Gareth E. Cleland, Salem, MA (US)

(21) Appl. No.: 15/963,223

(22) Filed: Apr. 26, 2018

Related U.S. Application Data
(60) Provisional application No. 62/491,787, filed on Apr. 28, 2017.

(51) Int. Cl. G01N 33/82; H01J 49/00; H01J 49/16

(52) U.S. Cl. CPC G01N 33/82; H01J 49/165; H01J 49/004

(57) ABSTRACT

Methods and kits for determining the concentration of vitamin D within a sample through the measurement of previtamin D corresponding to each vitamin D of interest. Also described herein are methods for the use of tandem mass spectrometry to make the measurement, creation of calibrators using, derivatization of the vitamin D samples of interest, and creation of calibrators for the same.

### CROSS REFERENCE TO RELATED APPLICATIONS

This application claims priority to U.S. Provisional Application No. 62/491,787, filed on Apr. 28, 2017, and entitled “Methods for Quantifying Total Vitamin D”, which is incorporated herein by reference in its entirety.

### FIELD OF THE TECHNOLOGY

The present disclosure relates to methods for determining the total concentration of vitamin D within a sample using mass spectrometry including using the sum of vitamin Ds and pre-vitamin Ds responses to quantify the total vitamin Ds content. The described methods show that the pre-vitamin D constitutes a significant portion of the total vitamin D concentration

### BACKGROUND

Vitamin D is a group of fat-soluble secosteroids which is present in some foods and available as a dietary supplement. Vitamin D promotes calcium absorption in the gut and maintains adequate serum calcium and phosphate concentrations to enable normal mineralization of bone and to prevent hypocalcemic tetany. Vitamin D is also needed for bone growth and bone remodeling. Without sufficient vitamin D, bones can become thin, brittle, or misshapen. Vitamin D sufficiency prevents rickets in children and osteomalacia in adults. Together with calcium, vitamin D also helps protect older adults from osteoporosis.

In human diets, two vitamin D secosteroids are of particular significance: vitamin D₃ (cholecalciferol) and vitamin D₂ (ergocalciferol). Also biologically active, and a key component to vitamin D₃ and vitamin D₂ are the isomerized precursors known as pre-vitamin D₃ and pre-vitamin D₂. Depending upon certain conditions, such as the presence of, or change in, metabolic state (e.g., enzymatic assisted conversion), as well as exposure to external stimuli (temperature, humidity, light stimuli such as UV radiation, etc.), the relative concentrations of pre-vitamin D and vitamin D present in a sample can change, thus impacting the chemical composition of the sample.

For quality control purposes, government regulations require that the concentration of vitamin D in e.g., a food sample be reported. See Food Labeling: Revision of the Nutrition and Supplement Facts Labels, 81 Fed. Reg. 33742 (May 27, 2016). The problem, however, is that many popular analytical methods systematically neglect the amount of pre-vitamin D present in a sample such that the concentration reported to the consumer may be inaccurate. Methods for accurately determining the concentration of Vitamin D, particularly as it relates to determining and comparing the amounts of pre-vitamin D within a sample, are needed.

Analytical methods have been used in the past to determine concentration of vitamin D. See e.g., Abernethy, G. A. Anal Bioanal Chem (2012) 403:1433; Gill, et al., J. of AOAC Intl. March/April 2015, 98:431-435; AOAC Official Methods 982.29, 992.26, 2012.11 and 2016.05. However, these methods have flaws or do not account for the vitamin D concentration variance resulting from the interconversion to or from pre-vitamin D. For example, the use of ultraviolet detection with liquid chromatography requires extensive sample preparation and clean up. See, e.g., AOAC Official Methods 982.29 and 992.26. Thus, it is not overly cost-effective and is not favorable for use in high-throughput analyses. Also, while the implementation of mass spectrometry reduced sample preparations and improved throughout, it was not used to account for interconversionary changes. See, e.g., AOAC Official Methods 2012.11 and 2016.05. Indeed, previous reports indicated that the amounts of pre-vitamin D could be disregarded as having no effect on the total concentration of Vitamin D.

### SUMMARY

Here, however, it has been shown that failing to account for the isomerization effects between pre-vitamin D and vitamin D could have implications of up to 20% variance. See e.g., FIG. 3, which shows the relative vitamin D3 content in samples with different heat history. The relative vitamin D3 content (in the total vitamin D3) can range from close to 100% to about 80% in samples undergoing different treatments. Indeed, the present methods have shown that ingredient labels on commercially-available food products do not accurately reflect the vitamin D concentration. See e.g., Table 7. There is therefore a need for more accurate and reproducible methods for determining the total concentration of vitamin D in a sample.

Provided herein are methods for determining the total concentration of vitamin D within a sample using mass spectrometry comprising using the sum of vitamin Ds and pre-vitamin Ds responses to quantify the total vitamin Ds content. The described methods show that the pre-vitamin D constitutes a significant portion of the total vitamin D concentration. See e.g., the exemplification section below. Additionally, failure to include pre-vitamin D resulted in a significant, difference between the total vitamin D concentration and what is listed on the label. See e.g., the exemplification section below.

Also provided are methods of determining the presence of, or the total amount of pre-vitamin Ds present in a sample. In some aspects, isotopically labelled standards of the pre-vitamin Ds can be used to construct calibration curves to assist in the determination of the vitamin Ds present in the sample.

Further provided are kits for determining the concentration of vitamin Ds in a sample using the disclosed methods.

### BRIEF DESCRIPTION OF THE FIGURES

FIG. 1 illustrates differences in peak areas for vitamin D3 and pre-vitamin D3 within room temperature and heated samples as obtained by mass spectrometry.

FIG. 2 illustrates the relative response for vitamin D3:PTAD and pre-vitamin D3:PTAD (over the isotopic labeled vitamin D₃ internal standard) by reaction time at temperature.

FIG. 3 illustrates the relative vitamin D₃ concentration in the total vitamin D₃ concentration (solid square), and the relative isotope labeled vitamin D₃ concentration in the total isotope labeled vitamin D₃ concentration (cross) in samples resulting from different heat treatments. The total vitamin D₃ concentration is the sum of the vitamin D₃ and the previtamin D₃. The total isotope labeled vitamin D₃ concentration is the sum of the isotope labeled vitamin D₃ and the isotope labeled previtamin D₃.

FIG. 4A illustrates a chromatographic separation and mass spectrometer signals for vitamin D from a mixture of standard solutions. FIG. 4B illustrates a chromatographic separation and mass spectrometer signals for vitamin D from a sample of infant formula.

FIG. 5 represents a calibration curve (the ratio of the total peak area over the internal standard's total peak area, vs the concentration ratio of the vitamin D₃ over the internal standard) for vitamin D₃.

FIG. 6 represents a calibration curve (the ratio of the total peak area over the internal standard's total peak area, vs the concentration ratio of the vitamin D₂ over the internal standard) for vitamin D₂.

### DETAILED DESCRIPTION

In one aspect, provided herein are methods of determining the total concentration of vitamin D within a sample using mass spectrometry, the method comprising (i) ionizing the sample to form precursor ions of the constituents present in the sample; (ii) mass selecting, from the first generation precursor ions, ions having a mass corresponding to vitamin D and pre-vitamin D, or a derivative thereof; (iii) fragmenting at least a portion of the mass-selected first generation product ions to produce second generation product ions of vitamin D and pre-vitamin D, or a derivative thereof; and (iv) determining the concentration of vitamin D from the response signals of the second generation product ions of vitamin D and pre-vitamin D. The methods can include one or more of the following embodiments.

In some embodiments, determining the concentration of vitamin D includes generating a calibration curve. The calibration curve can be, for example, a graph of total peak area against concentration, where the total chromatographic peak area is determined from the area of peaks on a chromatogram generated by measuring the response associated with a monitored mass-to-charge ratio over the course of the chromatographic elution. Alternatively, the calibration curve can be ratio of total peak area over concentration ratio, where ratio of total peak area is the ratio of (i) total peak area of a sample vitamin D to (ii) the total peak area of an internal standard vitamin D. The concentration ratio can be the ratio of concentration of the vitamin D in the sample to the concentration of the vitamin D in the internal standard. In some aspects, each of the total peak area of the sample vitamin D and the total peak area of the internal standard vitamin D can be adjusted to include the peak area associated with each corresponding pre-vitamin D peak. The corresponding pre-vitamin D peak can be adjusted by the use of a relative response factor, as described below, and the adjusted pre-vitamin D peak can be added to the vitamin D peak in order to generate a total vitamin D peak which includes both the vitamin D and the adjusted vitamin D.

In some embodiments, determining the concentration of vitamin D includes comparing the response signal of the second generation product ions to one or more internal standards. In some embodiments, comparing the response signal of the second generation product ions to one or more internal standards comprises measuring total peak area associated with both vitamin D and pre-vitamin D within the internal standard. In some embodiments, determining the concentration of vitamin D comprises determining relative response of one or more pre-vitamin D species within the sample as compared to corresponding vitamin D species within the sample. For example, the relative response of pre-vitamin D to vitamin D would reflect the ratio of signal associated with a given concentration of pre-vitamin D within the sample to the same concentration of vitamin D within the sample. One example in which relative response of pre-vitamin D to vitamin D may be significant, is where the yield of the derivatization reaction for pre-vitamin D and vitamin D differs. If, for example, the reaction of pre-vitamin D with derivative has a lower yield that the reaction of vitamin D with derivative, then when the derivatized pre-vitamin D and derivatized vitamin D are measured, the pre-vitamin D will be underrepresented relative to the vitamin D. This error can be corrected or minimized through the use of the relative response.

In some embodiments, determining the concentration includes finding a total peak area for one or more vitamin Ds present within the sample. Finding the total peak area includes e.g., calculating the sum of (i) peak area of one or more vitamin Ds and (ii) the product of peak area of the corresponding previtamin D isomer and a corresponding relative response factor.

In some embodiments, the internal standard used herein is an isotopically labeled form of vitamin D or pre-vitamin D, or a derivative thereof. In some embodiments, the internal standard is an isotopically labeled derivative of pre-vitamin D or vitamin D that has been modified from a reaction with 4-Phenyl-3H-1,2,4-triazole-3,5(4H)-dione (PTAD). In some embodiments, the internal standard is an isotopically labeled version of a compound having the formula:

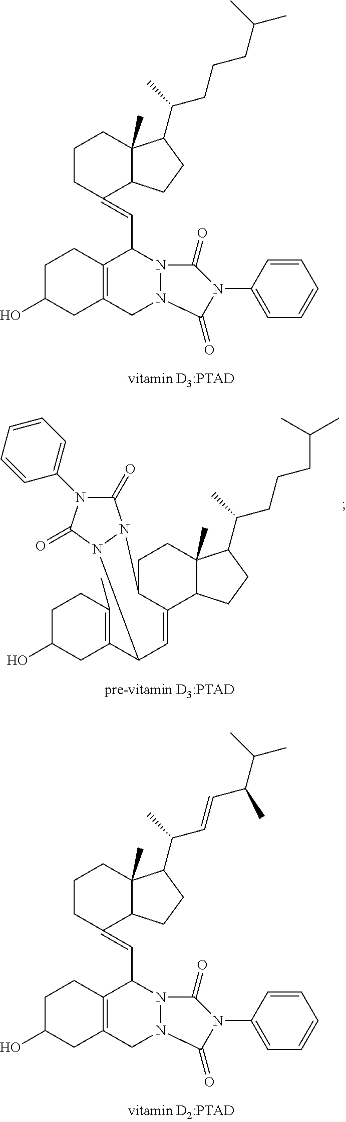 [SMILES: CC(C)CCC[C@@H](C)C1CCC2/C(=C/C3C4=C(CC(O)CC4)Cn4c(=O)n(-c5ccccc5)c(=O)n43)CCC[C@@]21C ; CC1=C(C2C=C3C(CC[C@@]4(C)C3CCC4[C@H](C)CCCC(C)C)n3c(=O)n(-c4ccccc4)c(=O)n32)CC(O)CC1 ; CC(C)[C@@H](C)/C=C/[C@@H](C)C1CCC2/C(=C/C3C4=C(CC(O)CC4)Cn4c(=O)n(-c5ccccc5)c(=O)n43)CCC[C@@]21C]

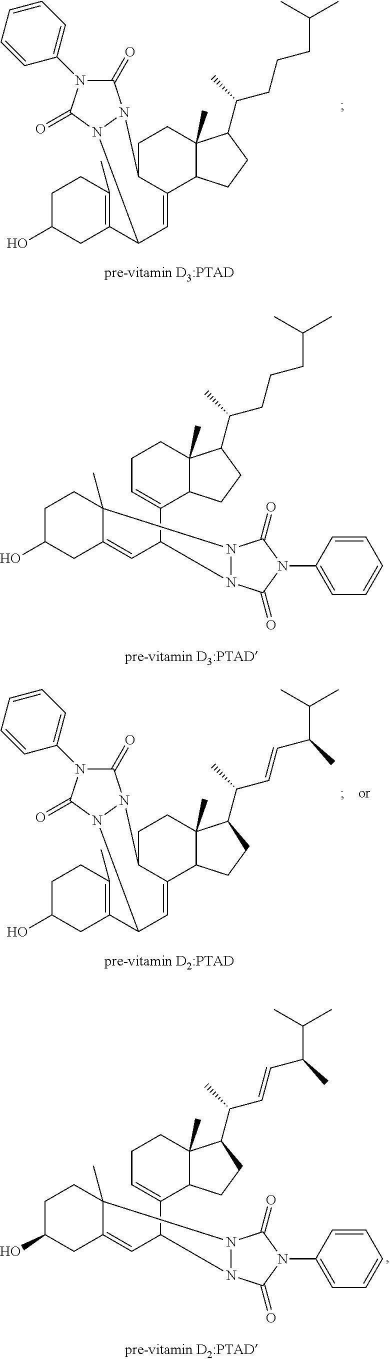

wherein one or more carbon or hydrogen atoms can be replaced by ²H or ¹³C.

In some embodiments, determining the concentration of vitamin D includes determining one or more relative response factors. Each of the one or more relative response factors can be determined from the response of at least a first calibrator and a second calibrator. In some embodiments, the second calibrator is a mixture of vitamin D and pre-vitamin D after heating. Heating can comprise heating the second calibrator e.g., above 50° C., above 55° C., above 60° C., above 65° C., or above 70° C. In some embodiments, the second calibrator can be heated at about 50° C., at about 55° C., at about 60° C., at about 65° C., at about 70° C., at about 75° C., at about 80° C., at about 85° C., at about 90° C., at about 95° C., at about 100° C., at about 105° C., at about 110° C., at about 115° C., at about 120° C., at about 125° C., at about 130° C., at about 135° C., at about 140° C., at about 145° C., or at about 150° C. In some embodiments heating comprises heating the second calibrator at about 75° C. Each of the foregoing values can also form the endpoint of a range, for example the second calibrator can be heated from about 110° C. to about 135° C. In some embodiments, heating comprises heating the second calibrator for about one hour. In some embodiments, the second calibrator can be heated for about 10 minutes, about 15 minutes, about 20 minutes, about 25 minutes, about 30 minutes, about 35 minutes, about 40 minutes, about 45 minutes, about 50 minutes, about 55 minutes, about 1 hour, about 75 minutes, about 90 minutes, about 105 minutes, about 2 hours, about 2.5 hours, about 3 hours, about 3.5 hours, about 4 hours, about 8 hours, about 12 hours, about 24 hours, about 48 hours, about 72 hours, or about 96 hours. Each of the foregoing values can also form the endpoint of a range, for example the second calibrator can be heated for about 75 minutes to about 90 minutes.

The temperature and time can be optimized for the conditions of the analysis. For example, a temperature can be selected which is at or below the boiling point of the solvent used in the second calibrator. The second calibrator can be refluxed. The time can also be adjusted based upon the temperature selected. Since the conversion between the pre-vitamin and vitamin compounds is temperature dependent, where a lower temperature is to be used, a longer time can be used. Conversely, if the time is to be reduced, the temperature can be increased, and so on.

In some embodiments determining the one or more response factors includes a first calibrator which is mixture of vitamin D and pre-vitamin D. In some embodiments, the concentration of vitamin D and pre-vitamin D of the second calibrator after heating is different than the concentration of vitamin D and pre-vitamin D in the first calibrator.

In some embodiments, the response factor can be determined by comparing the response (i.e., peak area) associated with each of vitamin D and previtamin D in the first calibrator and in the second calibrator. That is, the relative response factor can be calculated according to Formula I:

relative response factor=([D_{second calibrator}]−[D_{first calibrator}])/([preD_{first calibrator}]−[preD_{second calibrator}]) (Formula I)

The present study shows that one can achieve two samples with different concentrations of vitamin D and pre-vitamin D while maintaining the same total concentration of vitamin D and pre-vitamin D. The vitamin D and pre-vitamin D isomers are in equilibrium and interchange between each other. The equilibrium ratio of the two varies with temperature, with the vitamin D form being favored at lower temperatures and with the equilibrium shifting toward parity as temperature increases. In other words, as temperature increases, the concentration of pre-vitamin D within the sample increases and the concentration of vitamin D decreases. Where the same solution is used, the total concentration of vitamin D (i.e., vitamin D and pre-vitamin D) will be the same both before and after heating, however the proportions of vitamin D and pre-vitamin D will vary.

In some embodiments the total concentration of vitamin D is the total concentration of vitamin D₂ or the total concentration of vitamin D₃. For some applications, vitamins D₂ and vitamin D₃ can be the only forms of vitamin D that are of interest, and the sum of the concentration of vitamin D₂ and vitamin D₃ can be either the actual total concentration of vitamin D, or a satisfactory approximation of the actual total concentration of vitamin D. This may be the case, for example, in measurement of human or animal foods, or in nutritional supplements, where vitamin D₂ and vitamin D₃ are the predominate forms of vitamin D present. In some applications, other forms of vitamin D may also be of interest and can be measured, including D₄ (22-dihydroergocalciferol) and D₅ (sitocalciferol). As used herein, total concentration of vitamin D can particularly include the concentration of pre-vitamin D. That is, the total concentration of vitamin D₂ can include both the concentration of vitamin D₂ and the concentration of pre-vitamin D₂, and the concentration of vitamin D₃ can include both the concentration of vitamin D₃ and the concentration of pre-vitamin D₃. Additionally, the concentrations of pre-vitamin D₂ or pre-vitamin D₃ can be adjusted using the relative response factor, as described herein, in order to more accurately represent the actual concentration of pre-vitamin.

In some embodiments, the vitamin D and pre-vitamin D present in the sample are derivatized prior to ionization. In some embodiments, the vitamin D and pre-vitamin D are derivatized with 4-Phenyl-3H-1,2,4-trazole-3,5(4H)-dione (PTAD) prior to ionization.

In some embodiments, the mass selected ions correspond to a derivative of vitamin D having a formula selected from:

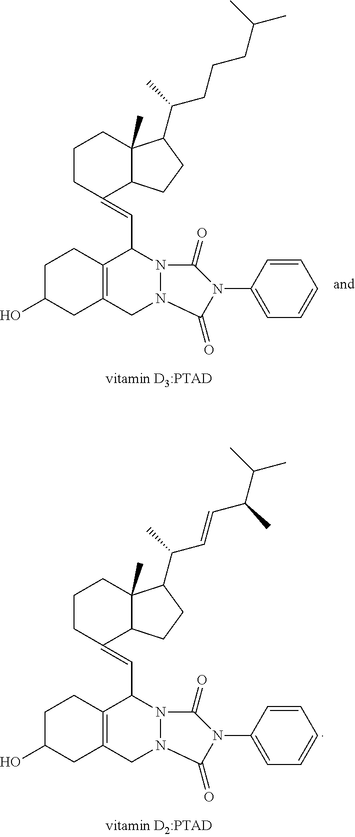 [SMILES: CC(C)CCC[C@@H](C)C1CCC2/C(=C/C3C4=C(CC(O)CC4)Cn4c(=O)n(-c5ccccc5)c(=O)n43)CCC[C@@]21C ; CC(C)[C@@H](C)/C=C/[C@@H](C)C1CCC2/C(=C/C3C4=C(CC(O)CC4)Cn4c(=O)n(-c5ccccc5)c(=O)n43)CCC[C@@]21C]

In some embodiments, the mass selected ions correspond to a derivative of pre-vitamin D having a formula selected from

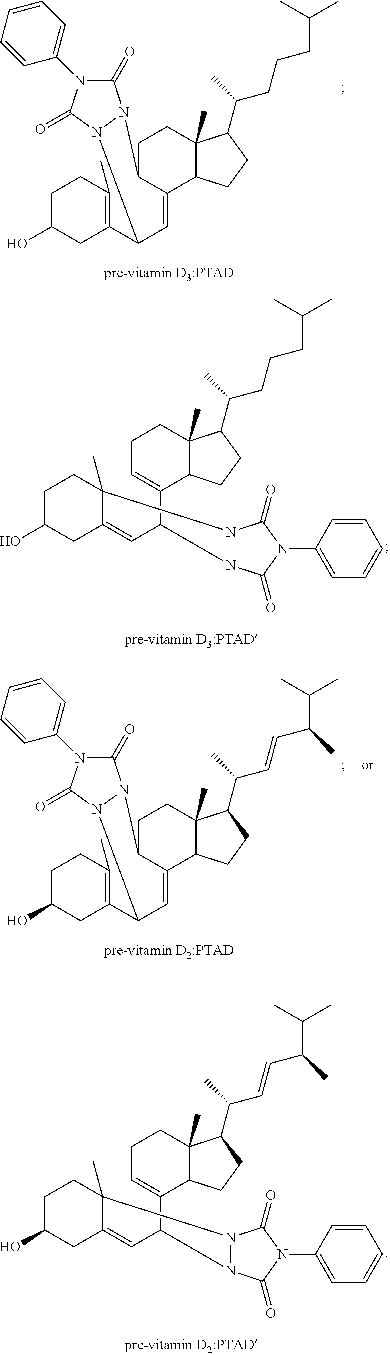

In some embodiments, the selection of second generation product ions for monitoring can be used to selected fragments characteristic of only one or of more than one derivative. The peaks corresponding to these derivatives can be summed in order to determine a total concentration.

In some embodiments, the disclosed sample can be ionized using an ion source selected from electrospray ionization, matrix assisted laser desorption/ionization, chemical ionization, atmospheric solids analysis ionization, atmospheric pressure vapor source, desorption electrospray ionization, and atmospheric pressure photoionization.

In some embodiments, the methods described herein can additionally include chromatographically separating the vitamin D and pre-vitamin D, or derivatives thereof. The chromatographic separation can be by liquid chromatography, high performance liquid chromatography, ultra-high performance liquid chromatography, or supercritical fluid chromatography, such as carbon dioxide-based chromatography. In some embodiments, chromatographically separating the vitamin D and pre-vitamin D, or derivatives thereof, comprises separating using a C18 column, such as for example a reverse-phase C18 nanoflow column

In some embodiments, the sample is a human or animal food, a human or animal dietary supplement, a pharmaceutical composition, a cosmetic composition, or a plasma or blood sample.

In some embodiments, provided herein is a method for determining the total concentration of vitamin D within a sample using electrospray ionization including, reacting the sample with 4-Phenyl-3H-1,2,4-triazole-3,5(4H)-dione (PTAD) to form vitamin D and pre-vitamin D derivatives selected from vitamin D₂:PTAD, pre-vitamin D₂:PTAD, vitamin D₃:PTAD, and pre-vitamin D₃:PTAD to form a derivatized sample; ionizing the derivatized sample to form precursor ions, ions having a mass corresponding to vitamin D₂:PTAD, pre-vitamin D₂:PTAD, vitamin D₃:PTAD, and pre-vitamin D₃:PTAD; fragmenting at least a portion of the mass-selected first generation product ions to produce second generation product ions; and determining the concentration of vitamin D₂, previtamin D₂, vitamin D₃, and previtamin D₃ from response of the second generation product ions. The method can include one or more of the above described embodiments.

In some embodiments, provided herein are kits for the determination of the concentration of vitamin D in a sample including a stable isotope labelled internal standard of vitamin D; a calibrator solution; and instructions for (i) ionizing the sample to form precursor ions of the constituents present in the sample; (ii) mass selecting, from the first generation precursor ions, ions having a mass corresponding to vitamin D and pre-vitamin D, or a derivative thereof; (iii) fragmenting at least a portion of the mass-selected first generation product ions to produce second generation product ions of vitamin D and pre-vitamin D, or a derivative thereof; and (iv) determining the concentration of vitamin D from the response signals of the second generation product ions of vitamin D and pre-vitamin D. The kits can include one or more of the above described embodiments.

### EXEMPLIFICATION

### General Methods of Preparation

Samples were prepared as follows: NIST 1849a reference material was purchased from NIST. Vitamin D₃ (cholecalciferol), vitamin D₂ (ergocalciferol), and PTAD were purchased form Sigma Aldrich Corp., St. Louis Mo. Stable isotope internal standard cholecalciferol (6, 19, 19-d3) was purchased from Cambridge Isotope Laboratories, Inc., Tewksbury, Mass. Food samples, such as infant formulas (milk and soy based), oatmeal, fish oil, vitamin D fortified milk power and vitamin D fortified chocolate were purchased in local grocery stores.

All standardized solutions were prepared in brown vials. Stock standard solutions of vitamin D₃ and D₂ were prepared in ethanol at about 1 mg/ml, respectively. The intermediate stock standard mix (#1) was prepared by mixing 10 μL of the individual stock standard solutions with 980 μL of acetonitrile to form 10 m/mL solution. This intermediate stock standard mix was further filtered with acetonitrile to form intermediate standard mixes at 1 m/ml (#2) and 0.1 μg/ml (#3) standard mix solutions. Internal standards (vitamin D₃-d3) solution at 10 m/ml was prepared by diluting the 100 m/ml ethanol solution with acetonitrile. The working standard solutions of vitamin D₃ and D₂ at concentrations from 1 pp to 500 ppb were prepared by mixing the appropriate intermediate standard mixes, the internal standard solutions, a acetonitrile. The concentration of the internal standard in these working standard solutions was kept at 50 ppb.

PTAD was dissolved in acetone at 10 mg/mL solution. It was further diluted in acetonitrile to form 1 mg/mL solution.

100 μL of these individual working standard solution were nitrogen-blow dried in brown vials and then mixed with 0.6 mL PTAD/acetonitrile solution (1 mg/mL). The mixture was vortexed for 30 seconds, and kept in room temperature for 40 minutes. Then 0.4 mL water was added to end the derivatization reaction. Standard solutions were filtered by 0.2 μm PTFE membrane before injection.

Samples were weighed (approximately 0.5 g) and spiked with internal standard. They were then mixed with water (4 mL) to form a homogeneous mixture by one minute of vortexing, 16 mL of pyrogallol ethanol solution (2 g/100 mL) was added and missed with vortexing (30 seconds), then 8 L of potassium hydroxide (KOH) water solution (50%) was added and vortexed for 30 seconds. These mixtures were capped and put into a water bath at 75° C. for one hour with occasional vortexing. After saponification for 1 hour, the mixtures were cooled to room temperature quickly in an ice-water bath. Liquid-liquid extraction was carried out with 12 ml of hexanes (12.5 mg/L). The upper layer (hexanes) was washed with water (8 mL) four times, with 1 minute vortexing and centrifugation (2500 rpm) carried out at each wash. Then 6 mL of the extract (hexanes layer) was nitrogen blow dried using a 30° C. heating block. The dried extracts were mixed with 0.6 mL PTAD (1 mg/mL in acetonitrile) for 40 minutes, then 0.4 mL water was added. The solution was filtered with 0.2 μm PTFE membrane before injection.

The chromatographic separation was performed using an Acquity™ UPLC high performance liquid chromatography system equipped with 2.1×50 mm ethylene bridged hybrid (BEH) C18 column with 1.7 μm particle size. The system and column are commercially available from Waters Technologies Corp., Milford, Mass. The column was operated at 40° C. with a 0.6 mL/min flow rate. A gradient mobile phase was used, with component A being water with 0.1% formic acid, and component B being acetonitrile with 0.1% formic acid, according to the following gradient in Table 1:

TABLE 1

| | % A (water, | % B (acetonitrile, | |
|---|---|---|---|
| Time | 0.1% formic acid) | 0.1% formic acid) | Curve |
| Initial | 80 | 20 | Initial |
| 0.25 | 80 | 20 | 6 |
| 2.75 | 0 | 100 | 6 |
| 6.5 | 0 | 100 | 6 |
| 6.6 | 80 | 20 | 6 |

The detector was a tandem mass spectrometry unit equipped with an electrospray ionization source operating in positive ion mode.

Multiple reaction monitoring (MRM) protocol is shown below in Table 2.

TABLE 2

| | | First | Second | | | |
|---|---|---|---|---|---|---|
| | | mass | Mass | Dwell | Cone | Collision |
| | Compound | (MS1) | (MS2) | (secs) | Voltage | Energy |
| 1 | D3:PTAD | 560.30 | 161.00 | 0.032 | 43 | 36 |
| 2 | D3:PTAD | 560.30 | 298.10 | 0.032 | 43 | 19 |
| 3 | PreD3:PTAD | 560.30 | 365.35 | 0.032 | 43 | 21 |
| 4 | PreD3:PTAD | 560.30 | 383.30 | 0.032 | 43 | 13 |
| 5 | SILD3:PTAD | 563.20 | 301.20 | 0.032 | 43 | 16 |
| 6 | SILPreD3:PTAD | 563.20 | 386.30 | 0.032 | 43 | 11 |
| 7 | D2:PTAD | 572.30 | 311.80 | 0.032 | 43 | 15 |
| 8 | D2:PTAD | 572.30 | 448.24 | 0.032 | 43 | 19 |
| 9 | PreD2:PTAD | 572.30 | 377.30 | 0.032 | 43 | 9 |
| 10 | PreD2:PTAD | 572.30 | 395.30 | 0.032 | 43 | 9 |

Additional mass spectrometry parameters were: capillary voltage of 1.20 k kV, source temperature of 150° C., desolvation temperature 500° C., cone gas flow of 0 L/hr, and desolvation gas flow of 1000 L/Hr.

As shown above, this example provides for identification of at least one characteristic fragment for a particular first generation ion mass which has been selected (in this case) for the derivatized vitamin D and pre-vitamin D to be measured. By contrast, this is beneficial because earlier UV based methods typically capture both vitamin and pre-vitamin concentration where the signal of both overlaps, without specific detector settings.

### Example 2

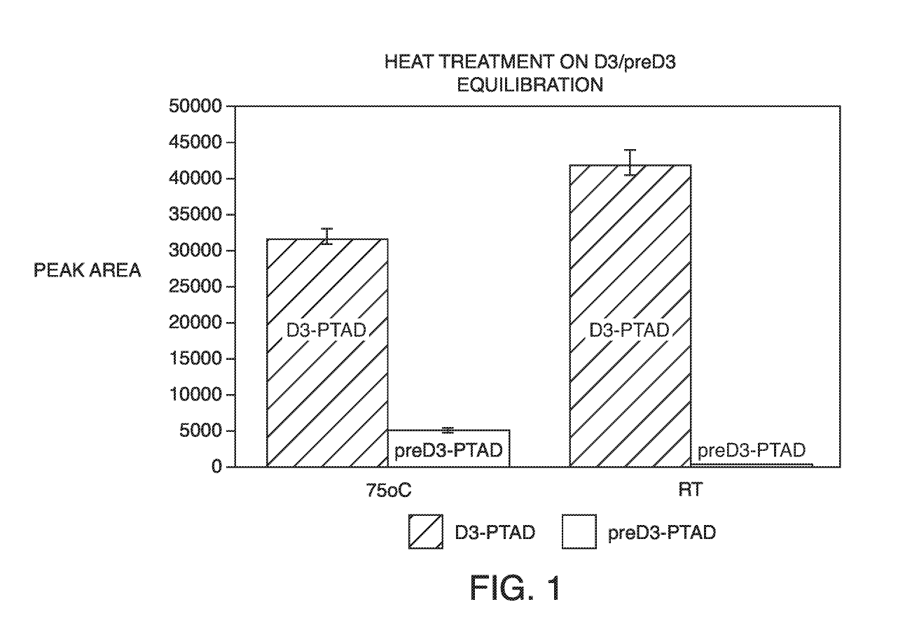

FIG. 1 shows the peak areas reported for each of vitamin D3:PTAD and previtamin D3:PTAD at 75° C. and when maintained at room temperature. FIG. 1 demonstrates that the pre-vitamin D₃:PTAD forms a significant portion of the total vitamin D:PTAD concentration within the sample heated to 75° C. before measurement. The prevalence of pre-vitamin D₃ in the heated portion represents a substantial change as compared to the room temperature sample. This example demonstrates that failing to account for pre-vitamin D₃ can lead to a significant underestimation of the total vitamin D count, especially where a sample is or has been heated. It is important to note that many sample preparation procedures, such as the saponification methods tests below specifically require heating, therefore increasing the pre-vitamin D₃ proportion as shown in FIG. 1.

The example further shows how the derivatization reaction can be optimized in order to achieve more accurate results for both vitamin D and pre-vitamin D within the sample by achieving optimal yield of derivatized vitamin and pre-vitamin.

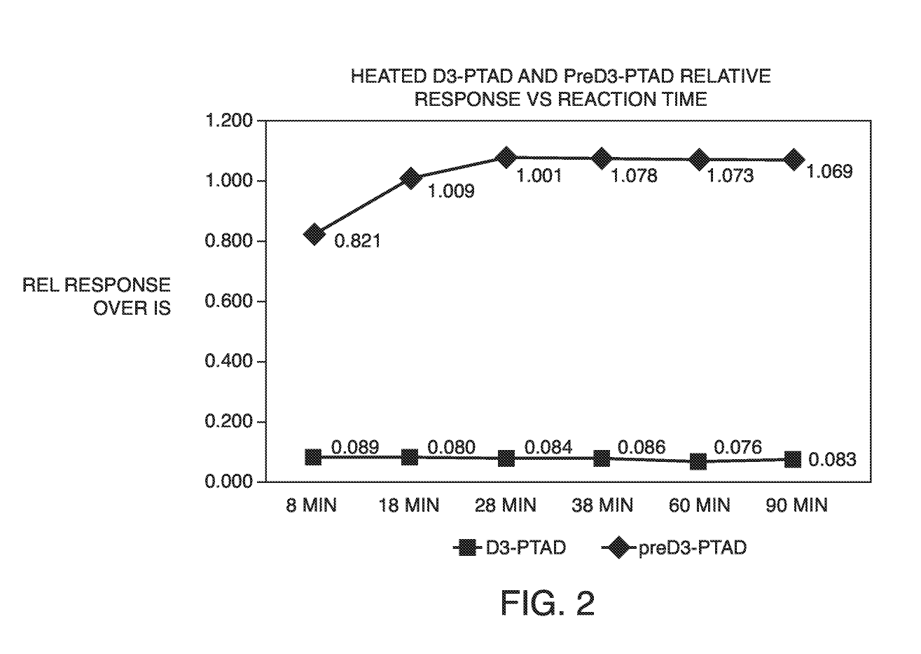

FIG. 2 shows the relative response as compared to the internal standard for vitamin D3:PTAD and previtamin D3:PTAD under heating over time, with sample measurements taken over time up to 90 minutes. FIG. 2 shows that the peak response for previtamin D3 occurred at about 40 minutes of heating. At the same point, vitamin D₃:PTAD concentration remained high. This result shows that about 40 minutes represents an optimal reaction time at 75° C. for vitamin D₃ derivatization with PTAD. This result can be extended to vitamin D₃ because the structural differences between vitamins D₂ and D₃ are limited and are located relatively far from the point of addition of the PTAD. Achieving optimal derivatization can increase the accuracy of the measurement, e.g., with regard to the pre-vitamin D, by providing a larger and therefore more easily measured signal.

### Example 3

Table 3 shows results from a comparison of determination of total vitamin D₂ and vitamin D₃ using two different analytical methods. Method A does not include the pre-vitamin D content in the calculations. Method B includes the pre-vitamin B content in the calculation as disclosed herein. Each method was used to analyze the concentration of D₃ and D₂ in a sample standard solution prepared at room temperature as compared to a sample treated at high temperature (75° C. for 1 hour).

TABLE 3
Comparison of Two Different Calculation Methods

| | Method A | | Method B | |
|---|---|---|---|---|
| | (Previtamin D content | | (Previtamin D content | |
| | is not used in | | is used in | |
| | calibration and calculation.) | | calibration and calculation.) | |
| (μg/g) | D₃ | D₂ | D₃ | D₂ |
| C-5 (Room | 9.200 | 9.190 | 9.543 | 9.561 |
| Temp.) | | | | |
| C-5 (High | 10.348 | 10.236 | 9.750 | 9.636 |
| Temp.) | | | | |
| Difference | 12% | 11% | 2% | 1% |

Note:
C-5 standard solution was prepared at RT. Then, a portion of C-5 was heated at 75° C. for 1 hour to form the High Temp sample. Average of four measurements.

As shown in Table 3, the vitamin D concentration calculated using Method B is less temperature dependent. When using the Method A calculation, the measurement for the standard sample increased on heating by 12% and 11% respectively. Conversely, Method B showed increases of only 2% and 1% respectively. The large variation in the determination by Method A results from the failure to account for pre-vitamin Ds within Method A.

Example 3 demonstrates a situation in which the failure to account for pre-vitamin Ds causes a higher calculated vitamin D concentration. This effect is attributable to the fact that Method A fails to include pre-vitamin D concentration with the internal standard measurement as well as within the sample measurement. The relative concentration of deuterated vitamin D and pre-vitamin D within the standard also changes with temperature. For this reason, the direction of the change in the reported total vitamin D concentration (i.e., over reporting vs. underreporting actual vitamin D concentration) depends on the relative shift of the equilibration between the vitamin D/pre-vitamin D for the internal standard (deuterated vitamin Ds) and for the analytes (vitamin Ds). Further unpredictability for calculation methods that do not account for pre-vitamin D concentration arises from the kinetics of the equilibrium interchange between vitamin D and pre-vitamin D. The equilibration can be relatively slow as compared to sample preparation time. Therefore, where the initial temperatures of the sample and the internal standard are different, the vitamin D within the sample and within the internal standard may not have reached the same equilibrium concentrations before the measurement is made. This problem is obviated in Method B, where both vitamin D and pre-vitamin D are measured. However, when the pre-vitamin D concentration is not taken into account, as demonstrated by Method A, the heating of the sample results in significant changes in the calculated total vitamin D concentration.

### Example 4

Table 4 shows results for Methods A and B as described in Example 3. Two samples were tested: one from high temperature saponification and one from room temperature saponification.

TABLE 4
Comparison of Two Different Calculation Methods

| | Method A | | Method B | |
|---|---|---|---|---|
| | (Previtamin D content | | (Previtamin D content | |
| | is not used in | | is used in | |
| | calibration and calculation.) | | calibration and calculation.) | |
| (μg/g) | D₃ | D₂ | D₃ | D₂ |
| X-1 (High | 0.303 | 0.191 | 0.299 | 0.189 |
| Temp. | | | | |
| Saponification) | | | | |
| X-1 (Room | 0.285 | 0.185 | 0.303 | 0.194 |
| Temp. | | | | |
| Saponification) | | | | |
| Difference | −6% | −3% | 1% | 3% |

Note:
High Temp saponification: 75° C. for 1 hour. Room Temp. saponification: RT, overnight. Average of three measurements.

Example 4 shows that the variation in concentration is also reflected where the high temperature is used in the saponification—i.e., during sample preparation—even if the sample is not heated immediately before injection. Method B achieves a result which is less temperature dependent. Here again, high temperature during the workup yields an increased result for both forms of vitamin D according to a traditional method, which shows variation of 6% and 3%, as compared to variation of 1% and 3% according to methods of the present technology. It may be appreciated that while low temperature favors the vitamin form over the pre-vitamin form, the rate of conversion is highly temperature defendant. For example, it has previously been shown that equilibrium concentration is reached after only seven minutes at 120° C., but after 30 days at 20° C. See Keverling-Buisman, J. A., et al., J. Pharm. Sci, 57: 1326-1329 (1968).

Example 4 demonstrates that vitamin D can be significantly underestimates by analytical methods having a high temperature saponification step unless pre-vitamin D is measured, as provided herein.

### Example 5

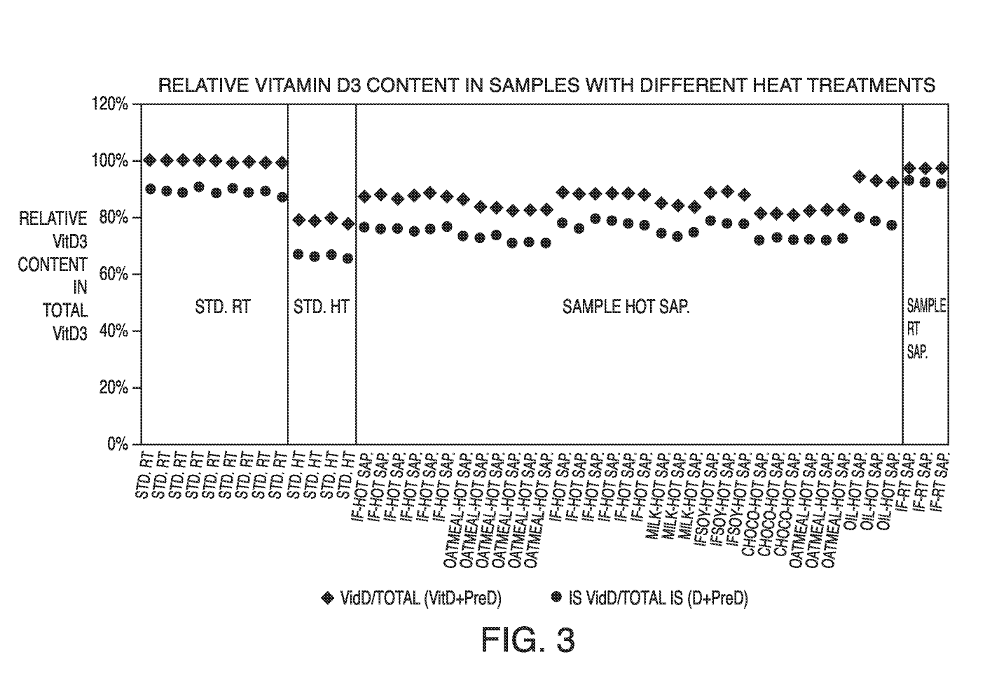

FIG. 3 shows the relative vitamin D₃ content as a percentage of total vitamin D₃ for a series of different standards and samples. Standards were either maintained at room temperature (Std-RT), were heated (Std-RT), were saponified at high temperature (infant formula, oatmeal, milk, soy-based infant formula, chocolate, and oil), or were saponified at room temperature (infant formula). In FIG. 3, a diamond indicates vitamin D₃ as a percentage of total vitamin D₃ (i.e., vitamin D₃ and pre-vitamin D₃) for the sample, while an “●” indicates the same value for the isotopically labelled internal standard. The results show that vitamin D₃ is a high percentage of the total for the room temperature samples (i.e., above about 95%), but falls to about 75% to 95% for the heated or high temperature saponified samples. In particular, the high temperature saponified samples generally exhibit only about 80 to 90% vitamin D₃. This shows that a total concentration measurement that disregards pre-vitamin D₃ underestimates the total vitamin D₃ concentration, and by as much as about 10 to 20%. High temperature saponification is often a preferred means of preparing a sample, and can be necessary to comply with established analytical practices and procedures used in industry.

The results in FIG. 3 show that the internal standard contains a large portion of pre-vitamin D₃ and—of particular significance—a different proportion of pre-vitamin D₃ than does the sample. Therefore, the failure to account for the pre-vitamin D₃ can also cause inaccuracy in the total reported vitamin D₃ concentration. Where that is the case, it is inappropriate to assume that the use of the internal standard alone will provide for adequate quantitation.

### Example 6

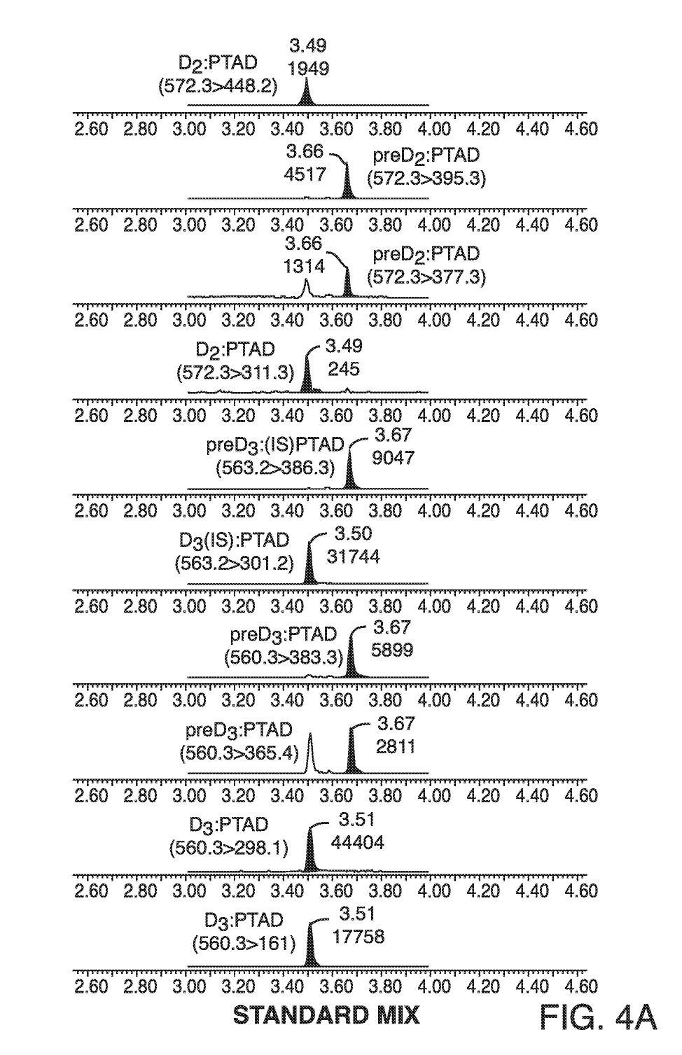

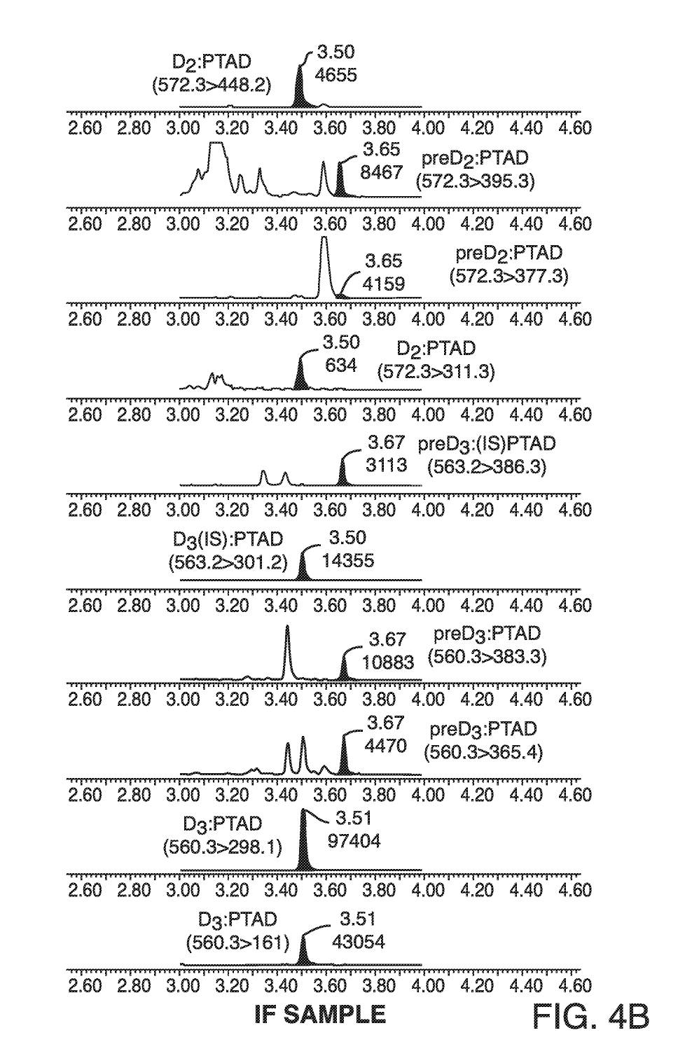

The mass spectrometry protocol as described in Table 1 was performed to generate chromatograms for each of the mass selection/fragmentation pairs monitored. FIG. 4A shows the chromatograms for the standard mixture, FIG. 4B shows the infant formula sample, and FIG. 7C shows the retention times for each compound. As shown in FIG. 4A-4B, the combination of chromatographic separation with mass spectrometry provides sharp peaks with little or no overlap with adjoining peaks. These features permit accurate quantitation. The chromatographic separation applied in Example 6 need not separate the vitamin D₃ and vitamin D₂ components, or the corresponding pre-vitamin components, and in fact, does not separate them, as shown by the retention times in Table 5, below. Instead, the tandem mass spectrometry analysis permits quantitation of these portions of the sample which would otherwise overlap using this chromatographic method.

TABLE 5

| | Retention Time (min) |
|---|---|
| D₃:PTAD | 3.50 |
| D₂:PTAD | 3.50 |
| SILD₃:PTAD | 3.50 |
| preD₃:PTAD | 3.67 |
| preD₂:PTAD | 3.67 |

### Example 7

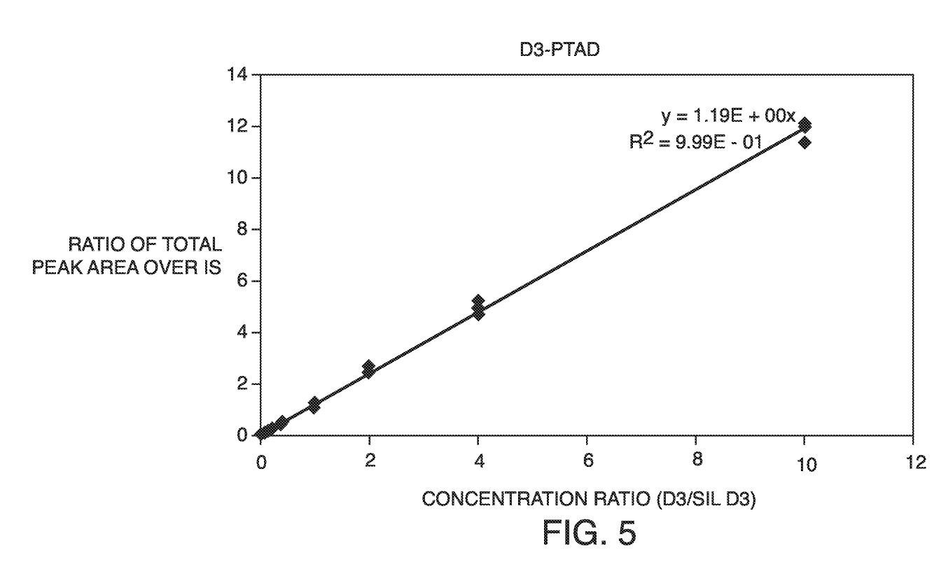

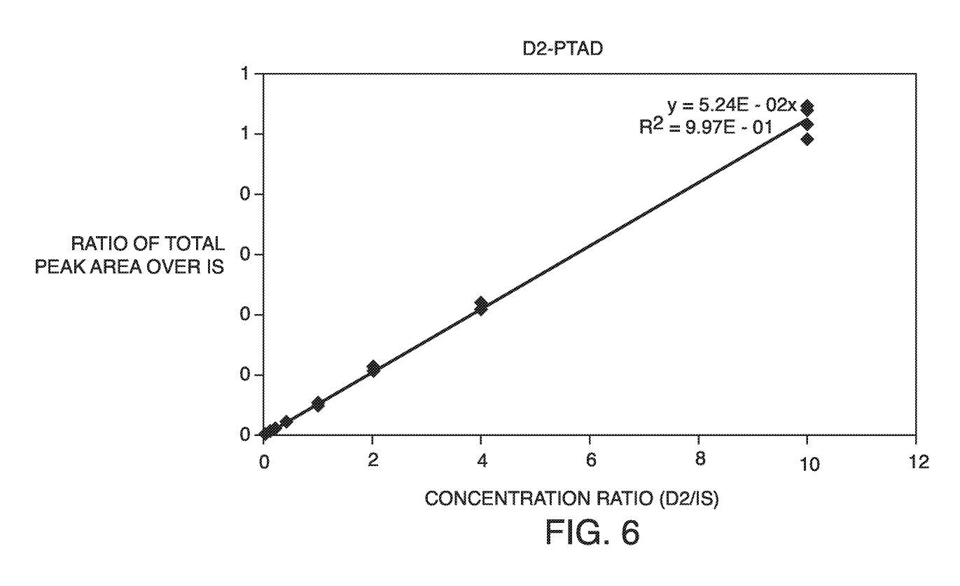

FIG. 5 shows a calibration curve for a vitamin D₃:PTAD derivative. FIG. 6 shows a calibration curve for a vitamin D₂:PTAD derivative. In both cases, a linear fit in shown on the graph. The fit shows that the data is well represented by a linear fit through zero, with an R² value of 0.999 for vitamin D₃ and of 0.997 for vitamin D₂. The limit of detection and limit of quantitation for the sample are shown in Table 6.

TABLE 6
Estimated LOD and LOQ in Vitamin D Measurement
in Samples and Standard Solutions

| | Infant Formula | | Oatmeal | | Solvent | |
|---|---|---|---|---|---|---|
| | D₃ | D₂ | D₃ | D₂ | D₃ | D₂ |
| LOD (mg/kg) | 0.01 | 0.009 | 0.003 | 0.006 | 0.00008 | 0.0007 |
| LOQ (mg/kg) | 0.04 | 0.03 | 0.01 | 0.02 | 0.0003 | 0.002 |

The ranges were 0.0004-0.2 mg/kg for vitamin D₃ and 0.002-0.2 mg/kg for vitamin D₂.

### Example 8

Table 7 shows experimental results for milk, infant formula (soy based), infant formula (milk based), energy bar, and canned tuna according to an embodiment of the present methods.

TABLE 7
Vitamin D₂ and Vitamin D₃ Concentration in Milk, Infant Formula (soy
based), Infant Formula (milk based), Energy Bar, and Canned Tuna

| | Non-fat Dry Milk | | | | | | | | | |
|---|---|---|---|---|---|---|---|---|---|---|
| | Fortified with | | Infant Formula | | Infant Formula | | | | | |
| | Vitamins A and D | | (Soy Based) | | (Milk Based) | | Energy Bar | | Canned Tuna | |
| Sample | Average | RSD | Average | RSD | Average | RSD | Average | RSD | Average | RSD |
| (μg/kg) | (μg/kg) | (%) | (μg/kg) | (%) | (μg/kg) | (%) | (μg/kg) | (%) | (μg/kg) | (%) |
| Vitamin D₃ | 104 | 1.4% | 83 | 6.2% | 70 | 6.8% | 0 | | 2 | 7.5% |
| Vitamin D₂ | 0 | | 0 | | 0 | | 11 | 5.6% | 0 | |
| Total Vitamin D | 104 | | 83 | | 70 | | 11 | | 2 | |
| Vitamin D | 109 | | 68 | | 68 | | 31 | | 10 | |
| content on label | | | | | | | | | | |
| Accuracy | 95% | | 122% | | 102% | | 36% | | 19% | |

Table 7 shows the results of measurements of the total Vitamin D content in food products. The vitamin D values on nutrition or supplement facts sheet of these foods were converted to numbers having units of μg/kg and listed in Table 7 for comparison. The determined vitamin D concentrations for milk and infant formulas were in agreement with the label claim for vitamin D values (less than 22% difference). The results for energy bar and canned tuna fish were significantly lower than the label claims. Accurately determining the actual concentration of vitamin D present in samples is important because regulations require labels to include a vitamin D concentration, thereof achieving an accurate vitamin D concentration is of considerable importance.

To emphasize the need to consider previtamin D in total vitamin D measurements, the same two sets of sample data were processed using two different methods of quantitation. A comparison of the methods is summarized in Table 8. In method A, total vitamin D was quantified without using the previtamin D peak area. This is the same data processing method that the standard method used. In method B, total vitamin D was quantified using both the previtamin D and the vitamin D peak areas in the calibration and the quantitation. As can be seen in Table 8, method A allowed 11-12% difference for the standards prepared at different conditions (high temperature, HT, vs. room temperature, RT) while method B only has 1-2% difference. For samples with different saponification conditions (HT saponification vs RT saponification), method A showed a larger difference (3-6%) than method B did (1-3%). The data in Table 8 shows that method B is less affected by the previtamin D concentration variation. Therefore, without measuring the previtimin D concentration, the total vitamin D analysis result can carry a large error that can be contributed to previtamin D formation during the manufacturing, transportation, or storage of food products.

TABLE 8
Comparison of two vitamin D methods in
the event of different heating history

| | Method A³ | | Method B³ | |
|---|---|---|---|---|
| | D₃ | D₂ | D₃ | D₂ |
| Standard (RT)¹ | 0.0092 | 0.0092 | 0.0095 | 0.0096 |
| Standards (HT)¹ | 0.0103 | 0.0102 | 0.0097 | 0.0096 |
| Difference between RT and HT | 12% | 11% | 2% | 1% |
| treatment | | | | |
| Sample (HT saponification)² | 0.303 | 0.191 | 0.299 | 0.189 |
| Sample (RT saponification)² | 0.285 | 0.185 | 0.303 | 0.194 |
| Difference between HT and RT | −6% | −3% | 1% | 3% |
| saponification | | | | |

Note:
¹Standard was split into two parts. One is kept at RT. The other was heated at 75° C. for 1 hour (HT)
²Samples from the same food product were split into two parts. One was saponified at 75° C. for 1 hour (HT saponification), the other was saponified at RT overnight (RT saponification)
³Method A does not include the previtamin Ds. Method B includes the previtamin Ds in the total vitamin Ds. The results are in mg/kg unit.

A range of food products can benefit from the present testing. For example, existing AOAC analytical methods that could be replaced with methods according to the present disclosure include the analysis of milk, dairy products, oils and fats, cereals, pre-mixes, baby food, infant formula, adult nutritional formula, feeds, poultry feed and supplements, and pet food.

### Example 9

Accuracy and recovery were also analyzed. Accuracy of vitamin D₃ measurement was made using a NIST 1849a reference material. Table 9 show that the present method yielded an overall accuracy of 102.6%.

TABLE 9

| | 1 | | 2 | | 3 | | Average | | | | | |
|---|---|---|---|---|---|---|---|---|---|---|---|---|
| | Mean | SD | Mean | SD | Mean | SD | Mean | SD | RSD | Ref. Values | | Accuracy |
| D₃ (mg/kg) | 0.116 | 0.003 | 0.107 | 0.002 | 0.118 | 0.003 | 0.114 | 0.003 | 2.4% | 0.111 | 0.017 | 102.6% |

The average shows 0.114 mg/kg vitamin D₃ with a standard deviation of 0.003, as compared to a references value of 0.111 mg/kg with a standard deviation of 0.017. Each of 1, 2, and 3 represent separate measurements of the NIST sample. Within each measurement, triplicate analysis were conducted.

Recovery was analyzed using infant formula and oatmeal samples, as shown in Table 10.

TABLE 10

| | |
|---|---|
| Infant Formula | Oatmeal |

| | D₃ | D₂ | D₃ | D₂ |
|---|---|---|---|---|
| Blank (μg/kg) | 0.116 | 0.030 | 0.000 | 0.000 |
| Spike level 3 (0.02 mg/kg) | N/A | N/A | 100% | 102% |
| Spike level 2 (0.09 mg/kg) | 116% | 98% | 110% | 117% |
| Average | 116% | 98% | 105% | 110% |

The present studies show that, contrary to previous expectations, the concentration of previtamin D can represent a significant and variable percentage of the total vitamin D concentration (i.e., the concentration of both vitamin D and pre-vitamin D). The present technology provides methods and kits for accurately determining the total vitamin D concentration within a sample.

Although vitamin D exists in both pre-vitamin D and vitamin D forms, prior analytical methods in common use did not differentiate between the two forms. In the case of ultraviolet-based detectors, the absorbance spectra for the vitamin D and previtamin D forms can be sufficiently similar (i.e. overlapping) that the absorbance measurement will essentially yield a total vitamin D measurement, or at least an approximate total vitamin D measurement. However, in the case of mass spectrometry, the fragments associated with each vitamin D and each corresponding previtamin D differ. For example, vitamin D₃:PTAD registers fragments at 161.00 and 298.10, whereas previtamin D3:PTAD registers fragments at 365.35 and 383.30. Thus, analysis of fragments associated with each vitamin D may not be assumed to include the corresponding previtamin.

While some prior mass spectrometry analysis have recognized that previtamin concentration may be excluded in mass spectrometry measurements, they have assumed that the previtamin D concentration is negligibly small and remains approximately constant. For example, prior studies have assumed that the pre-vitamin F concentration was less than about 5%. Therefore the studies either assumed that the total concentration tracked the vitamin D concentration (because the ratio of previtamin D to vitamin D was assumed to be essentially constant).

FIG. 2 shows that, in fact, pre-vitamin D concentration can be a significant proportion of a sample. As shown by Examples 3 and 4, failure to account for pre-vitamin D not only leads to underestimating vitamin D concentration, but also leads to inconsistency in measurements where temperate varies. Example 8 demonstrates that these problems can be reflected in vitamin D concentrations reported on labels of commercial food products. By contrast, measurements made according to the present method yield better reproducibility and greater accuracy. See, e.g., Examples 3, 4, and 7.

1. A method for determining the total concentration of vitamin D within a sample using mass spectrometry, the method comprising
ionizing the sample to form precursor ions of the constituents present in the sample;
mass selecting, from the first generation precursor ions, ions having a mass corresponding to vitamin D and pre-vitamin D, or a derivative thereof;
fragmenting at least a portion of the mass-selected first generation product ions to produce second generation product ions of vitamin D and pre-vitamin D, or a derivative thereof; and
determining the concentration of vitamin D from the response signals of the second generation product ions of vitamin D and pre-vitamin D.

2. The method of claim 1, wherein determining the concentration of vitamin D comprises generating a calibration curve.

3. The method of claim 1, wherein determining the concentration of vitamin D comprises comparing the response signal of the second generation product ions to one or more internal standards.

4. The method of claim 3, wherein comparing the response signal of the second generation product ions to one or more internal standards comprises measuring total peak area associated with both vitamin D and pre-vitamin D within the internal standard is measured.

5. The method of claim 1, wherein determining the concentration of vitamin D comprises determining relative response of one or more pre-vitamin D within the sample as compared to corresponding vitamin D within the sample.

6. The method of claim 1, wherein determining the concentration comprises finding a total peak area for each of one or more vitamin D within the sample.

7. The method of claim 6, wherein finding a total peak area comprises calculating the sum of (i) peak area of a vitamin D and (ii) the product of peak area of a corresponding pre-vitamin D and a corresponding relative response factor.

8. The method of claim 3, wherein the internal standard is an isotopically labeled form of vitamin D, pre-vitamin D, or a derivative thereof.

9. The method of claim 3, wherein the internal standard is an isotopically labeled form of vitamin D, and pre-vitamin D, or a derivative thereof having a formula selected from

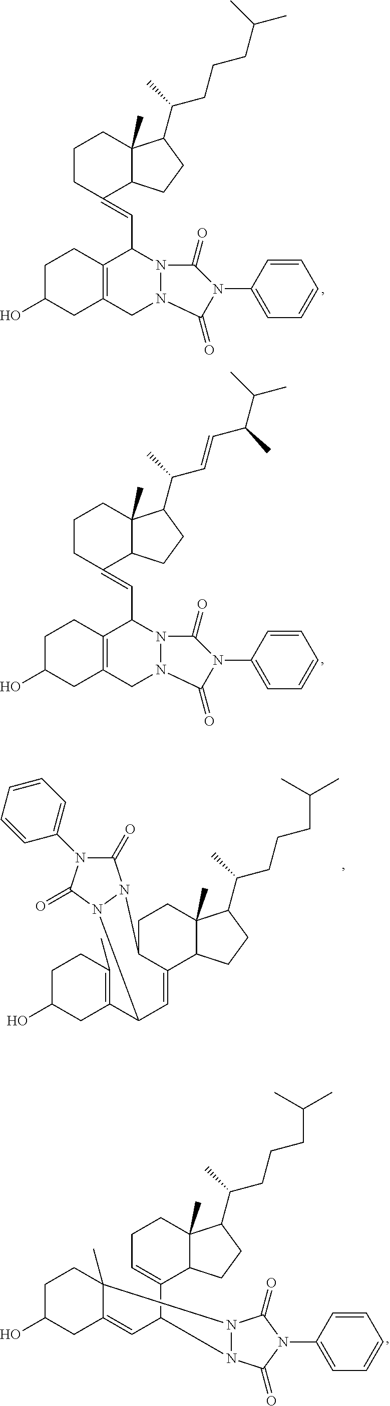

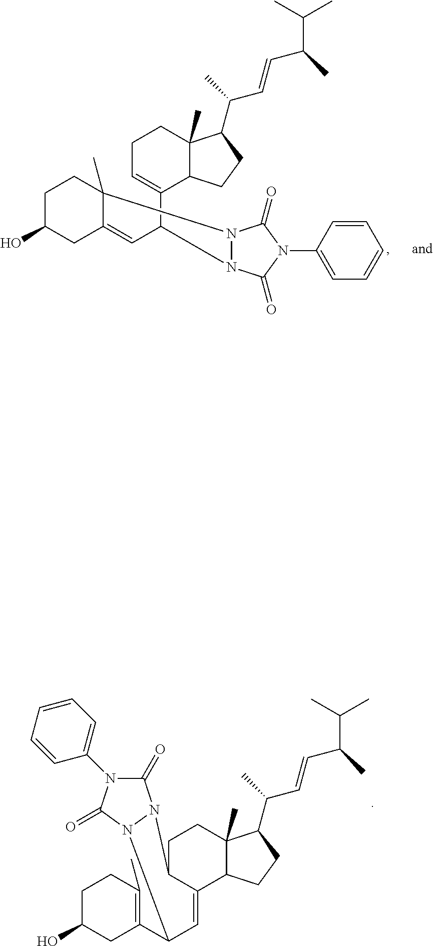

10. The method of claim 3, wherein the internal standard is an isotopically labeled form of vitamin D, pre-vitamin D, or a derivative thereof having at least one isotopic group selected from ²H and ¹³C.

11. The method of claim 1, wherein determining the concentration of vitamin D comprises determining one or more relative response factors.

12. The method of claim 11, wherein each of the one or more relative response factors are determined from the response of at a first calibrator and a second calibrator.

13. The method of claim 12, wherein the second calibrator is a mixture of vitamin D and pre-vitamin D after heating.

14. The method of claim 13, wherein heating comprises heating the second calibrator above 50° C.

15. The method of claim 13, wherein heating comprises heating the second calibrator from 60 to 80° C.

16. The method of claim 13, wherein heating comprises heating the second calibrator at about 75° C.

17. The method of claim 13, wherein heating comprises heating the second calibrator for about one hour.

18. The method of claim 13, wherein the first calibrator is a mixture of vitamin D and pre-vitamin D.

19. The method of claim 13, wherein the concentration of vitamin D and pre-vitamin D of the second calibrator after heating is different than the concentration of vitamin D and pre-vitamin D in the first calibrator.

20. The method of claim 1, wherein the total concentration of vitamin D is the total concentration of vitamin D₂ or the total concentration of vitamin D₃.
